SEQUENCE LISTING (1 sequence)

SEQ ID NO: 1 - DNA, 25 bp, Artificial Sequence
  source: /note=“Description of Artificial Sequence Synthetic oligonucleotide”
ttttttttttttttttttttttttt

(19) United States
(12) Patent Application Publication
(10) Pub. No.: US 2023/0220376 A1
(43) Pub. Date: Jul. 13, 2023

(54) HIGH THROUGHPUT LINKING OF MULTIPLE TRANSCRIPTS

(71) Applicant: ATRECA, INC., San Carlos, CA (US)

(72) Inventors: Sean CARROLL, San Carlos, CA (US); Gary WITHEY, San Carlos, CA (US); Sergey BOYARSKIY, San Carlos, CA (US)

(21) Appl. No.: 17/759,091

(22) PCT Filed: Jan. 22, 2021
(86) PCT No.: PCT/US2021/014631

§ 371 Date: Jul. 19, 2022

Related U.S. Application Data
(60) Provisional application No. 62/964,319, filed on Jan. 22, 2020.

(51) Int. Cl. C12N 15/10

(52) U.S. Cl. CPC C12N 15/1075; C12N 15/1068

(57) ABSTRACT

Provided are high throughput methods for physically linking cDNA molecules derived from mRNA molecules expressed by the same cell, and libraries of linked cDNA molecules produced by the methods. The methods comprise reverse transcribing mRNA from a single cell in a first container to produce cDNA molecules, and linking the cDNA molecules in a second container. The methods unexpectedly produced libraries of cDNA molecules with an increase in the number of molecules that are correctly linked to other molecules derived from the same cell.

### CROSS-REFERENCE TO RELATED APPLICATIONS

This application is a U.S. National Phase of PCT/US2021/014631, filed Jan. 22, 2021, which claims priority benefit of U.S. Provisional Application No. 62/964,319, filed Jan. 22, 2020, each of which is incorporated by reference in its entirety for all purposes.

### BACKGROUND

There have been a number of methods developed in recent years to achieve high-throughput capture of natively-paired immune repertoire sequences: heavy-light chain pairs in the case of B-cells and alpha-beta chain pairs in the case of T-cells. Information gleaned from the resulting datasets can provide unique insights into the inner workings of the human immune response and enable more informed approaches to developing therapeutics and vaccines, as examples.

One category of such methods involves the massively-parallel isolation of individual immune cells in micro-containers (e.g. droplets), subsequent lysis of cells within those containers, and capture of mRNA transcripts onto poly-T capture beads via the poly-A tail of the mRNA strands. Often, beads are then recovered from their containers with mRNA attached via hybridization. These beads are then washed, individually re-encapsulated into secondary micro-containers, the desired transcripts (heavy and light chain or alpha and beta chain) reverse-transcribed into cDNA, and finally those cDNA sequences are amplified and linked together into single amplicons that comprise both the heavy and light chain sequence or alpha and beta chain sequence from the cell of origin. Subsequent sequencing of linked amplicons can generate 1,00,000+ unique linked immune cell receptor sequences from a sample with minimal hands-on time from the operator. Additionally, linked amplicon fragments can be further manipulated into a display format (eg phage display, yeast, display) for interrogation of the immune repertoire for sequences that bind specific targets (eg, cells, specific proteins of interest).

A deficiency in these approaches exists, namely the propensity for mispairing and loss of transcripts that are consequences of the non-covalent attachment of mRNA transcripts to capture beads. Because cell transcripts are not covalently linked to the bead, there are multiple opportunities for cell transcripts to end up on the incorrect bead and thus lose the connection to the single cell. One example of such lost connection occurs in the intervening period between the two encapsulation steps mentioned above and detailed in the descriptions below. During this time between microcontainers, the bead sample must be treated with extreme care: washing solutions must be carefully tuned in terms of ionic strength, pH, and presence of surfactant and RNases, the sample must be kept cold at all times, and washing steps must be performed as rapidly as possible in order to minimize the extent of transcript loss, shuffling or otherwise random hybridization of transcripts to capture beads, and degradation of the relatively delicate mRNA transcripts.

Loss of transcripts can occur as a result of dehybridization of mRNA from the capture bead, or degradation of the mRNA transcripts themselves. Dehybridization can be caused by insufficient ionic strength or presence of certain surfactants or other reagents in the washing solution, failure to keep the bead solution cold at all times, or simply a prolonged time interval between the extraction of beads from the first container and re-encapsulation in the second container. Degradation of mRNA transcripts can be caused by exposure to RNases through contamination or by improper pH of the washing solutions. The consequence of degradation or dehybridization is the lack of sensitivity and accuracy of correct immune cell sequence pairing.

Mispairing occurs as a result of mRNA transcripts binding randomly to capture beads while outside of their original containers. This can be caused by any of the conditions mentioned above. In addition, there will nearly often be some excess of transcripts in the original containers that remain free in solution since the capture rate is nearly always less than 100% or because the beads are saturated with transcript. Once removed from their original containers, these excess free transcripts have opportunity to bind at random to other capture beads, and this random binding will also lead to mispairing.

The present disclosure describes a solution to the above identified problems with current methods.

### BRIEF SUMMARY

Described herein are methods for high-throughput linking of multiple transcripts that are highly sensitive and practically eliminates the sources of transcript loss and mispairing described above. Also described are methods for producing libraries of physically linked amplicons that were derived from the same single cell. The methods provide the unexpected advantage of increasing the percentage of amplicons that are correctly linked to amplicons from the same cell in the library.

In one aspect, a method for producing two or more linked nucleic acid molecules from a single cell is provided, the method comprising:
(i) isolating a single cell in a first container, and lysing the single cell to release mRNA molecules;
(ii) reverse transcribing the mRNA molecules to produce cDNA molecules in the first container; and
(iii) linking the cDNA molecules derived from the single cell in step (ii) in a second container,
thereby producing linked nucleic acid molecules.

In some embodiments, the first container comprises one or more solid supports attached to an oligonucleotide comprising a sequence complementary to a portion of the mRNA molecules. In some embodiments, the mRNA molecules are attached to the oligonucleotide via binding to the complementary sequence. In some embodiments, the reverse transcribing comprises extending the oligonucleotide with a reverse transcriptase to produce the cDNA molecules.

In some embodiments, the oligonucleotide is attached to the solid support by a linker. In some embodiments, the linker is located between a surface of the solid support and the sequence complementary to a portion of the mRNA molecules.

In some embodiments, the linker is a photocleavable linker. In some embodiments, the cDNA molecules are released from the solid support by exposing the photocleavable linker to light. In some embodiments, the linker is cleaved by ultraviolet (UV) light. In some embodiments, the cDNA molecules are released from the solid support in the second container. In some embodiments, the cDNA molecules are released from the solid support by exposing the photocleavable linker to light in the second container.

In some embodiments, the cDNA molecules from step (ii) above are covalently linked to the solid supports. In certain embodiments, each of the one or more solid supports is isolated (or dispersed into) in a different second container prior to step (iii).

In some embodiments, 1 to 20 solid supports are present in the first container. In some embodiments, an average of 3, 4 or 5 solid supports are present in the first container. In some embodiments, an average of 15 solid supports are present in the first container.

In some embodiments, the solid support is a bead or particle. In some embodiments, the solid support is a spherical particle having a diameter of 1 to 20 micrometers. In some embodiments, the solid support has an average diameter between 5 and 10 micrometers.

In some embodiments, linking the cDNA molecules in step (iii) comprises amplifying and linking the cDNA molecules by overlap extension PCR. In some embodiments, the overlap extension PCR comprises amplifying the cDNA molecules using one or more internal primers comprising a biotin tag. In some embodiments, cDNA molecules comprising the biotin tag are removed after the linking step. In some embodiments, the overlap extension PCR comprises amplifying the cDNA molecules using one or more external primers chemically modified to resist nuclease degradation. In some embodiments, the one or more external primers are chemically modified to include phosphorothioate bonds. In some embodiments, the cDNA molecules are contacted with a 5′-exonuclease after the linking step. The 5′-exonuclease can digest and degrade any molecules that do not contain a chemically modified external primer on both ends. In some embodiments, the cDNA molecules are released from the solid support prior to amplifying and linking the cDNA molecules.

In some embodiments, the single cell is an immune system cell, such as a B cell, a memory B cell, an activated B cell, a blasting B cell, a plasma cell, a plasmablast, a T cell, or a natural killer T (NKT) cell.

In some embodiments, the mRNA molecules encode a heavy chain variable region and a light chain variable region.

In certain embodiments, the cDNA molecules encode a cognate pair of heavy and light chain variable regions. In some embodiments, the cDNA molecules encode a cognate pair of T cell receptor alpha and beta chains.

In some embodiments, the first and/or second container comprises a partition, an aqueous droplet in an emulsion, a microvesicle, a tube, or a well in a multiwell plate.

In some embodiments, the droplet is 2 to 500 micrometers in diameter.

In some embodiments, the method further comprises digesting the mRNA following step (ii). In some embodiments, the mRNA is digested in the first container, or between steps (ii) and (iii).

In another aspect, a method for producing a library of linked nucleic acid molecules is described, the method comprising:
a) isolating a plurality of single cells in a plurality of first containers, where the first containers comprise a single cell;
b) lysing the single cells to release mRNA molecules in the first container;
c) reverse transcribing the mRNA molecules to produce cDNA molecules derived from single cells in the first container;
d) linking the cDNA molecules from step (c) in a second container;
e) combining the linked cDNA molecules from step (d) to produce a library of linked nucleic acid molecules.

In some embodiments, step (d) comprises amplifying and linking the cDNA molecules by overlap extension PCR. In some embodiments, the overlap extension PCR comprises amplifying the cDNA molecules using one or more internal primers comprising a biotin tag. In some embodiments, cDNA molecules comprising the biotin tag are removed after step (d). In some embodiments, the overlap extension PCR comprises amplifying the cDNA molecules using one or more external primers chemically modified to resist nuclease degradation. In some embodiments, the one or more external primers are chemically modified to include phosphorothioate bonds. In some embodiments, the cDNA molecules are contacted with a 5′-exonuclease after step (d).

In some embodiments, the single cells are B cells, and the percentage of heavy chain variable regions that are correctly paired with the cognate light chain variable regions in the library is increased compared to a method where steps (c) and (d) are performed in the same container.

In some embodiments, the single cells are T cells, and the percentage of T cell receptor alpha chains that are correctly paired with the cognate T cell receptor beta chains in the library is increased compared to a method where steps (c) and (d) are performed in the same container.

In some embodiments, the single cells are NKT cells, and the percentage of T cell receptor alpha chains that are correctly paired with the cognate T cell receptor beta chains in the library is increased compared to a method where steps (c) and (d) are performed in the same container.

In another aspect, a method for producing two or more linked nucleic acid molecules from a single cell is described, the method comprising:

(i) isolating a single cell in a first container, and lysing the single cell to release mRNA molecules;
(ii) hybridizing the mRNA molecules to a capture oligonucleotide attached to a solid support, wherein the capture oligonucleotide comprises a sequence complementary to a portion of the mRNA sequence;
(iii) reverse transcribing the mRNA molecules to produce cDNA molecules attached to the solid support in the first container;
(iv) linking the cDNA molecules derived from step (iii) in a second container,
thereby producing linked nucleic acid molecules.

In some embodiments, step (iv) comprises amplifying and linking the cDNA molecules by overlap extension PCR. In some embodiments, the overlap extension PCR comprises amplifying the cDNA molecules using one or more internal primers comprising a biotin tag. In some embodiments, cDNA molecules comprising the biotin tag are removed after step (iv). In some embodiments, the overlap extension PCR comprises amplifying the cDNA molecules using one or more external primers chemically modified to resist nuclease degradation. In some embodiments, the one or more external primers are chemically modified to include phosphorothioate bonds. In some embodiments, the cDNA molecules are contacted with a 5′-exonuclease after step (iv).

In some embodiments, the capture oligonucleotide further comprises a linker positioned between the solid support and the sequence complementary to a portion of the mRNA sequence. Thus, when the capture oligonucleotide is extended by reverse transcriptase to produce cDNA, the cDNA is covalently attached to the capture oligonucleotide, and the cDNA is thereby attached to the surface of the solid support via the linker.

In any of the embodiments described herein, the linker can be cleaved, releasing the cDNA molecules from the solid support prior to the step of amplifying and linking the cDNA molecules into a single amplicon.

In any of the embodiments described herein, linking the cDNA molecules can comprise amplifying and linking the cDNA molecules by overlap extension PCR. In some embodiments, the overlap extension PCR comprises amplifying the cDNA molecules using one or more internal primers comprising a biotin tag. In some embodiments, molecules comprising the biotin tag are removed after the overlap extension PCR step. The molecules comprising the biotin tag can be removed, for example, by contacting the molecules with streptavidin linked to a solid support, such as a bead or magnetic bead, and separating the molecules comprising the biotin tag that bind to streptavidin from the unbound molecules that do not comprise a biotin tag. In some embodiments, the overlap extension PCR comprises amplifying the cDNA molecules using one or more external primers that are chemically modified to resist nuclease degradation. In some embodiments, the one or more external primers are chemically modified to include phosphorothioate bonds. In some embodiments, the cDNA molecules are contacted with a 5′-exonuclease after the overlap extension PCR step to digest and degrade molecules that do not contain a chemically modified external primer at both ends. Removal of the molecules comprising the biotin tag and/or degradation of non-linked, single chain molecules before further amplification provides the advantage of increasing the yield and correct pairing of the final linked product.

### BRIEF DESCRIPTION OF THE DRAWINGS

FIG. 1 shows a schematic of a state of the art method and two embodiments of the current disclosure. In all three cases, Step 1 involves lysis of the cell within the container followed by hybridization of mRNA template to the capture beads. In the case of the state of the art method, mRNA template remains hybridized to the capture beads during Step 2. In Step 3, the emulsion is broken and the beads are washed. It is at this stage where some mRNA template is often lost from the bead, shuffled between beads, or captured at random from the contents of another container. In Step 4, the bead is re-encapsulated in a second container, and then reverse transcription into cDNA and amplification and linkage between target cDNAs can be achieved. In one embodiment of the current disclosure, Step 2 involves reverse transcription of the mRNA targets directly onto the capture beads followed by digestion of the original mRNA template. In Step 3, beads are extracted from their containers and washed without risk of losing the cDNA targets since they are covalently bound to the beads. In Step 4, the beads are re-encapsulated into a secondary container where the cDNA can be amplified and the desired products linked together in single amplicons. In an alternative embodiment of the current disclosure, Step 2 involves reverse transcription of mRNA targets into cDNA directly on the capture beads. In Step 3, beads are extracted from their containers, washed, and the mRNA template is digested away. There is no risk of loss of cDNA target because the cDNA target is covalently bound to the bead. In Step 4, the beads are re-encapsulated into a secondary container where the cDNA can be amplified and the desired products linked together in single amplicons.

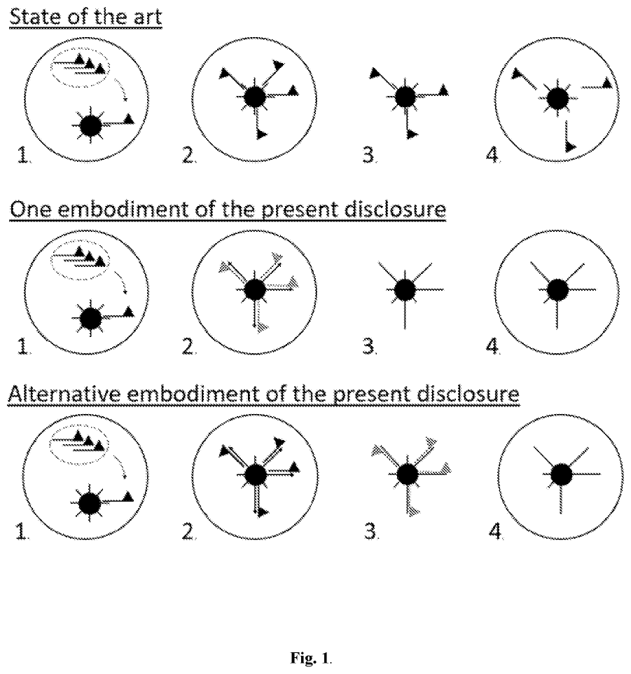

FIG. 2 shows a schematic drawing of a microfluidic droplet chip with oil input channels in a flow-focusing configuration for droplet formation and the following aqueous input channels: (1) cells in a suspension buffer and (2) capture beads in lysis/reverse transcription (RT) mix. Multiple different embodiments are possible with the various components (cells, beads, lysis mix, RT mix) combined or split among different microfluidic channels that all converge to merge their components at ratios to comprise the ultimate mix that is desired in the droplets. Barcoded beads and cells are loaded into aqueous droplets as described by a Poisson distribution. The average values (lambdas) of beads per droplet and cells per droplet are a function of the concentration of those components in their input streams. The droplet is the reaction container in which cell lysis and the reverse transcription reaction takes place.

FIG. 3 shows representative linking strategies to conjugate capture DNA oligonucleotides to a solid support. A copper-free click chemistry approach uses azide-modified oligonucleotides and DBCO-functionalized solid support. In carboxyl-amine coupling, an amine-modified oligo is conjugated with a carboxylic acid-functionalized solid support. A non-covalent but strong bond can also be achieved by coupling biotinylated oligonucleotides to solid support that has been modified with streptavidin molecules.

### DETAILED DESCRIPTION

### Terminology

The term “derived from” refers to a compound or molecule that is produced directly or indirectly from another molecule. The term “derived from a single cell” refers to a molecule that is directly isolated from a single cell, or a molecule that is synthesized from a molecule that was isolated from a single cell. If the molecule isolated from the single cell is a nucleic acid molecule, the term includes molecules comprising a complementary, or reverse-complementary, sequence to the isolated nucleic acid molecule. For example, a cDNA molecule is derived from a single cell if the cDNA was synthesized from an mRNA template molecule isolated from the single cell.

The term “solid support” refers to a composition comprising a solid surface that is suitable for binding or attaching a nucleic acid thereto.

The terms “polynucleotide(s)” and “nucleic acid(s)” refers to DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in nucleotide sequences by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods described herein.

As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with a polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Complementary sequences include base-pairing of a region of a polynucleotide comprising a first nucleotide sequence to a region of a polynucleotide comprising a second nucleotide sequence over the length or a portion of the length of one or both nucleotide sequences. Such sequences can be referred to as “complementary” with respect to each other herein. However, where a first sequence is referred to as “substantially complementary” with respect to a second sequence herein, the two sequences can be complementary, or they may include one or more, but generally not more than about 5, 4, 3, or 2 mismatched base pairs within regions that are base-paired. For two sequences with mismatched base pairs, the sequences will be considered “substantially complementary” as long as the two nucleotide sequences bind to each other via base-pairing.

“Complementary” sequences, as used herein, may also include, or be formed entirely from, non-Watson-Crick base pairs and/or base pairs formed from non-natural and modified nucleotides, in as far as the above embodiments with respect to their ability to hybridize are fulfilled. Such non-Watson-Crick base pairs includes, but are not limited to, G:U Wobble or Hoogstein base pairing.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information web-site.

Identical sequences include 100% identity of a polynucleotide comprising a first nucleotide sequence to a polynucleotide comprising a second nucleotide sequence over the entire length of one or both nucleotide sequences. Such sequences can be referred to as “fully identical” with respect to each other herein. However, in some aspects, where a first sequence is referred to as “substantially identical” with respect to a second sequence herein, the two sequences can be fully complementary, or they may have one or more mismatched nucleotides upon alignment. In some aspects, where a first sequence is referred to as “substantially identical” with respect to a second sequence herein, the two sequences can be fully complementary, or they may be at least about 50, 60, 70, 80, 81, 82, 83, 84, 85, 86, 87, 88, 89, 90, 91, 92, 93, 94, 95, 96, 97, 98, or 99% identical to each other.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;”

sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

The term “messenger RNA” or “mRNA” refers to an RNA that is without introns and that can be translated into a polypeptide.

The term “cDNA” refers to a DNA that is complementary or identical to an mRNA, in either single stranded or double stranded form.

The term “amplicon” refers to the amplified product of a nucleic acid amplification reaction, e.g., RT-PCR.

The term “hybridize” refers to a sequence specific non-covalent binding interaction with a complementary nucleic acid. Hybridization may occur to all or a portion of a nucleic acid sequence. Those skilled in the art will recognize that the stability of a nucleic acid duplex, or hybrids, can be determined by the Tm. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

As used herein, “region” refers to a contiguous portion of the nucleotide sequence of a polynucleotide. Examples of regions are described herein an include identification regions and sample identification regions. In some aspects, a polynucleotide can include one or more regions. In some aspects, regions can be coupled. In some aspects, regions can be operatively coupled. In some aspects, regions can be physically coupled.

As used herein, “variable region” refers to a variable nucleotide sequence that arises from a recombination event, for example, it can include a V, J, and/or D region of an immunoglobulin or T cell receptor sequence isolated from a T cell or B cell of interest, such as an activated T cell or an activated B cell.

As used herein “B cell variable immunoglobulin region” refers to a variable immunoglobulin nucleotide sequence isolated from a B cell. For example, a variable immunoglobulin sequence can include a V, J, and/or D region of an immunoglobulin sequence isolated from a B cell of interest such as a memory B cell, an activated B cell, or plasmablast.

As used herein, the term “native pair” or “cognate pair” refers to immunoglobulin genes encoding heavy and light chain variable regions expressed by the same B cell, or T cell receptor (TCR) genes encoding alpha and beta chains of the TCR expressed by the same T cell.

As used herein “identification region” refers to a first nucleotide sequence (e.g., a unique barcode sequence) that can be coupled to second, distinct nucleotide sequence to allow, for example, later identification of the second nucleotide sequence.

As used herein, “barcode” or “barcode sequence” refers to any unique sequence that can be coupled to at least one nucleotide sequence to allow, for example, later identification of the at least one nucleotide sequence.

As used herein “immunoglobulin region” refers to a contiguous portion of nucleotide sequence from one or both chains (heavy and light) of an antibody.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

The term “container” refers to an enclosed or partially enclosed space that is suitable for performing the molecular biology reactions described herein, and includes a partition, an aqueous droplet in an emulsion, a microvesicle, a tube, or a well in a multiwell plate.

The term “capture oligonucleotide” refers to an oligonucleotide comprising a nucleic acid sequence that is complementary to at least a portion of another nucleic acid sequence. For example, the capture oligonucleotide can include a sequence that is complementary to at least a portion of an mRNA sequence present in a sample.

The term “about,” when modifying a numerical value herein, encompasses normal variation encountered by those of ordinary skill in the art. Thus, the term “about” includes plus or minus 0.1%, 0.5%, 1.0%, 2%, 5% or 10% variation in the modified numerical value. All ranges provided herein include the endpoints and all values in between the endpoints to the first significant digit.

### Methods for Linking Transcripts

Described herein are methods for linking transcripts from cells that are highly sensitive and practically eliminates the sources of transcript loss and mispairing that occur with current methods known in the art. This high sensitivity and increased accuracy is accomplished by reverse transcribing an mRNA template into cDNA that is covalently attached to a solid support (for example, a capture bead) while the solid supports remain in their original containers. In one aspect, the reverse transcription (RT) step is performed in a separate container from the amplification and linking steps. In some embodiments, the mRNA transcripts are destroyed by digestion before the solid supports leave the first container. The methods can unexpectedly increase the sensitivity of subsequent PCR steps and as a consequence only the sequences that are present in their original containers are amplified and linked together. This innovative step carries the primary benefit of significantly improving pairing fidelity and sensitivity compared to existing methods. For example, the inventors unexpectedly found that performing the reverse transcription step in the first container resulted in a significant increase in the percentage of linked cDNAs derived from the same cell (e.g., natively paired cDNAs) compared to performing the RT step after the solid supports were removed from the first container and before adding the solid supports to the second container, or performing the RT step after the solid supports were added to the second container. The methods also provide a secondary benefit in that the process is more robust and can be paused after the solid supports are extracted from their original containers and before they are added to secondary containers. This secondary benefit offers the advantage of greater workflow flexibility.

The methods described herein can include, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Green & Sambrook, et al., Molecular Cloning: A Laboratory Manual (4th Edition, 2012); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B (1992); Current Protocols in Molecular Biology (2002—; Wiley; Online ISBN: 9780471142720; DOI: 10.1002/04711142727); Current Protocols in Immunology (2001—; Wiley; Online ISBN: 9780471142737; DOI: 10.1002/0471142735).

In one aspect, methods for producing two or more linked nucleic acid molecules are described. The methods described herein differ from methods currently used in the art in that cell lysis and the reverse transcription reaction are performed in a first container (container 1) using oligo-dT primers conjugated to a solid support (such as a bead), resulting in cDNA that is covalently linked to the solid support in the first droplet, whereas the PCR amplification reaction linking the cDNAs is performed in a second container (container 2). The advantages provided by the instant methods include (i) less contamination (for example, cross-contamination of transcripts from different samples binding to solid supports from other containers, resulting in linked cDNA molecules are no longer derived from the same sample) because cDNA is permanently and covalently linked to the solid support, and (ii) increased sensitivity of the RT reaction. The Examples provide representative embodiments of the methods. In one representative embodiment, the steps of the method include individual encapsulation of cells into emulsion droplets, in-droplet lysis of the cell(s), reverse transcription to produce cDNA, incorporation of the cDNA into droplet 2, and PCR in droplet 2 to link together cDNA molecules. In some embodiments, the linked cDNA molecules encode immunoglobulin heavy and light chains derived from a single cell.

In some embodiments, the nucleic acid molecules were originally present in a biological sample, such as a cell. In some embodiments, the nucleic acid molecules encode immune system proteins, such and IgG heavy and light chain variable regions, or T cell receptor alpha and beta chains. In some embodiments, the nucleic acid molecules encode native pairs (also referred to as “cognate pairs”) of IgG heavy and light chain variable regions, or T cell receptor alpha and beta chains.

In some embodiments, the method comprises (i) isolating a single cell in a first container, and lysing the cell to release nucleic acid molecules, (ii) generating a complementary copy of the nucleic acid molecules in the first container; and (iii) linking the complementary copies of the nucleic acid molecules in a second container, thereby producing linked nucleic acid molecules. In some embodiments, the nucleic acid molecules are RNA molecules. In some embodiments, the nucleic acid molecules are messenger RNA (mRNA) molecules.

Thus, in some embodiments, the method comprises (i) isolating a single cell in a first container, and lysing the cell to release mRNA molecules, (ii) reverse transcribing the mRNA molecules to produce cDNA molecules in the first container; and (iii) linking the cDNA molecules in a second container, thereby producing linked nucleic acid molecules. In some embodiments, steps of the method occur in the following order: (i), followed by (ii), followed by (iii).

In some embodiments, the cDNA molecules in step (iii) are derived from the mRNA molecules present in a single cell. In other words, the mRNA molecules present in a single cell are released from the single cell when the cell is lysed, and reverse transcribed into cDNA using methods known in the art. For example, the mRNA molecules can be contacted with an oligonucleotide primer comprising a nucleic acid sequence complementary to a portion of the mRNA molecules under conditions that promote hybridization of the oligonucleotide primer to the complementary sequence in the mRNA, and the primer can be extended by contacting the mRNA/oligonucleotide heteroduplex with an enzyme having reverse transcriptase activity.

In some embodiments, the first container comprises one or more solid supports attached to an oligonucleotide comprising a sequence complementary to a portion of the mRNA molecules. The oligonucleotide can hybridize to the complementary to a portion of the mRNA molecules, such that the mRNA molecules are attached to the oligonucleotide via binding to the complementary sequence. In some embodiments, the mRNA is reverse transcribed by extending the oligonucleotide with a reverse transcriptase to produce cDNA molecules, such that the cDNA molecules are covalently linked to the solid supports.

In some embodiments, the oligonucleotide attached to the solid support functions to hybridize to mRNA transcripts (i.e., “captures” mRNA transcripts, therefore alternatively referred to as a “capture oligonucleotide”) and serves as a primer for the initial reverse transcription reaction to reverse transcribe mRNA molecules into cDNA molecules (via extension of the oligonucleotide primer by reverse transcriptase). In some embodiments, a linker is located between the solid support surface and the oligonucleotide, such that the oligonucleotide is indirectly attached to the solid support surface via a linker. In some embodiments, the linker is a photocleavable linker. In some embodiments, the linker can be cleaved by ultraviolet (UV) light.

Following the production of solid supports attached to cDNA molecules in the first container, the solid supports are removed from the first container and transferred to a second container. In some embodiments, the mRNA template hybridized to the cDNA can be digested with enzymes prior to removing the solid support(s) from the first container. Thus, in some embodiments, the RNA template is destroyed prior to removing the solid support from the first container. While not being bound by theory, destroying the RNA template before performing the linking step may provide the advantage of reducing cross-contamination of transcripts from different samples binding to solid supports from other containers, such that linked cDNA molecules are no longer derived from the same sample. In the context of immunoglobulin variable regions, such cross-contamination would result in linked cDNAs that do not encode native pairs (also called cognate pairs) of heavy and light chain polypeptides.

In some embodiments, a thermostable RNase is used to digest the RNA template. In some embodiments, the thermostable RNase is RNase H. In one embodiment, the thermostable RNase is kept minimally active during the RT reaction, and then the temperature is increased to promote ribonuclease activity and extensively digest the RNA template.

In some embodiments, the mRNA digestion step is performed in the original container. In some embodiments, the mRNA digestion step is performed after the solid support is extracted from the original container and before re-encapsulation in the secondary container. In some embodiments, the mRNA digestion step is performed after the reverse transcription step. In some embodiments, the mRNA digestion step is performed after the reverse transcription step and before the amplification and/or linking step. In some embodiments, the mRNA transcripts are not intentionally destroyed, and persist during the washing steps and are encapsulated in the second container.

The solid supports can be washed to remove cellular materials, RNA and enzymes after the supports are removed from the first containers and before they are added to the second containers. After the solid supports are transferred to the second container, the cDNA molecules can be physically linked. In some embodiments, the cDNA molecules are amplified before being physically linked. In some embodiments, the cDNA molecules are amplified and physically linked in the same reaction, for example, by using overlap extension polymerase chain reaction (PCR) (“oePCR”). In some embodiments, the cDNA molecules are physically linked by joining the molecules to each other, for example by contacting the molecules with a ligase. In some embodiments, the cDNA molecules are physically linked by fusion of homologous ends using a Gibson reaction or a one-step PCR plus ligation reaction.

In some embodiments, each solid support from the first container is added to a different second container, such that the one or more solid supports from the first container are dispersed into one or more second containers, and each second container contains a single solid support. Thus, in some embodiments, each of the one or more solid supports extracted from the first container is added to a different (distinct) second container prior to the linking step, such that each second container contains a single solid support.

Removal of Single-Chain Fragments after Overlap-PCR for Increased Pairing Fidelity

The presence of single chain fragments from the overlap-PCR step can interfere with subsequent amplification and cloning of paired heavy and light chains, leading to mispairing of heavy and light chains. Minimizing single chain fragments before amplification can greatly increase yield and pairing fidelity of the final product. Thus, in another aspect, a method by which non-paired fragments are differentiated from correctly paired, overlapped product, and removed from the system is provided. In some embodiments, the method comprises introducing differential primers during the overlap-PCR reaction.

In some embodiments, the differential primers comprise the internal primers used to amplify the single chains, but the primers are not present in the final overlap-PCR product. In some embodiments, the differentiating factor is a tag that can be used to help remove any single-chain fragments left over from the overlap-PCR step.

For example, in some embodiments, the internal primers can be modified with a 5′ molecular tag such as a biotin tag. A streptavidin system such as magnetic streptavidin beads can be used to remove any DNA molecules left over after the overlap-PCR reaction which contain the biotin tag. Because the correctly paired, dual heavy and light chain linked overlapped fragments will no longer contain the biotinylated molecules, the desired correctly paired and linked heavy and light PCR fragments will remain while single-chain contaminating fragments can be removed with the streptavidin beads.

Alternatively, the outside primers amplifying the final overlapped product can be modified to include a differentiating factor. In some embodiments, the differentiating factor comprises a chemical modification. In some embodiments, the outside primers can be modified to resist depletion or degradation, for example when both outside primers are present on the molecule. In some embodiments, the outside primers can be chemically modified to resist nuclease or 5′-exonuclease degradation. Thus, in some embodiments, the outside primers can be modified to include phosphorothioate bonds in the backbone by inclusion of locked bases. The mixture of linked paired and single-chain molecules can be treated with a 5′-exonuclease prior to further amplification. Thus, only molecules with modified external primers on both ends (e.g., the linked heavy and light chains) would be resistant to exonuclease degradation. On the other hand, single chain molecules that contain only one modified external primer would not be resistant to exonuclease degradation, and can be digested with a 5′-exonuclease prior to further amplification, thus removing them from the reaction to reduce mispairing.

### Methods for Producing Libraries of Linked Nucleic Acid Molecules

In another aspect, the methods produce a library of linked nucleic acid molecules. In some embodiments, the method comprises:

a) isolating or distributing a plurality of single cells in a plurality of first containers, where the first containers comprise a single cell;
b) lysing the single cells to release mRNA molecules into the first container;
c) reverse transcribing the mRNA molecules to produce cDNA molecules in the first container;
d) linking the cDNA molecules in a second container; and
e) combining the linked cDNA molecules to produce a library of linked nucleic acid molecules.

In some embodiments, the single cells are B cells, and the percentage of heavy chain variable regions that are correctly paired with the cognate light chain variable regions in the library is increased compared to a method where the reverse transcribing and linking steps are performed in the same container.

In some embodiments, the single cells are T cells, and the percentage of T cell receptor alpha chains that are correctly paired with the cognate T cell receptor beta chains in the library is increased compared to a method the reverse transcribing and linking steps are performed in the same container.

In some embodiments, the single cells are NKT cells, and the percentage of T cell receptor alpha chains that are correctly paired with the cognate T cell receptor beta chains in the library is increased compared to a method where steps (c) and (d) are performed in the same container.

In some embodiments, cDNA molecules attached to the solid supports are released or cleaved from the solid support surface prior to the amplification (e.g., PCR) step in the second container. The inventors have unexpectedly found that the yield of product (e.g., number of heavy and light chain pairs recovered) and the product purity (e.g., proportion of natively-paired heavy and light chains) can be increased if the cDNA molecules are released from the solid support prior to performing overlap extension PCR that links the cDNA molecules together into a single amplicon. In some embodiments, the yield is increased by at least 5%, 10%, 15% or more compared to methods where the amplification step is performed without releasing the cDNA molecules from the solid support surface. In some embodiments, the purity is increased by at least 10%, 20%, 30%, 40%, 50%, 60%, 70%, 80%, 90%, 100%, 110%, 120%, 130%, 140%, 150% or more compared to methods where the amplification step is performed without releasing the cDNA molecules from the solid support surface.

In some embodiments, solid supports encapsulated in the second container are attached to cDNA molecules using a linker, as described herein. In some embodiments, the linker is cleaved to release the cDNA molecules before initiating the amplification (PCR) step. For example, in some embodiments, the linker is a photocleavable linker, and the linker is exposed to light comprising a wavelength capable of cleaving the linker, thereby releasing the cDNA molecules from the solid support surface. In some embodiments, the photocleavable linker is exposed to ultraviolet (UV) light (365 nM) for a time period suitable to cleave the linker (e.g., 5-10 minutes). Following cleavage of the linker, the cDNA molecules can be amplified, e.g., by PCR.

### Linkers

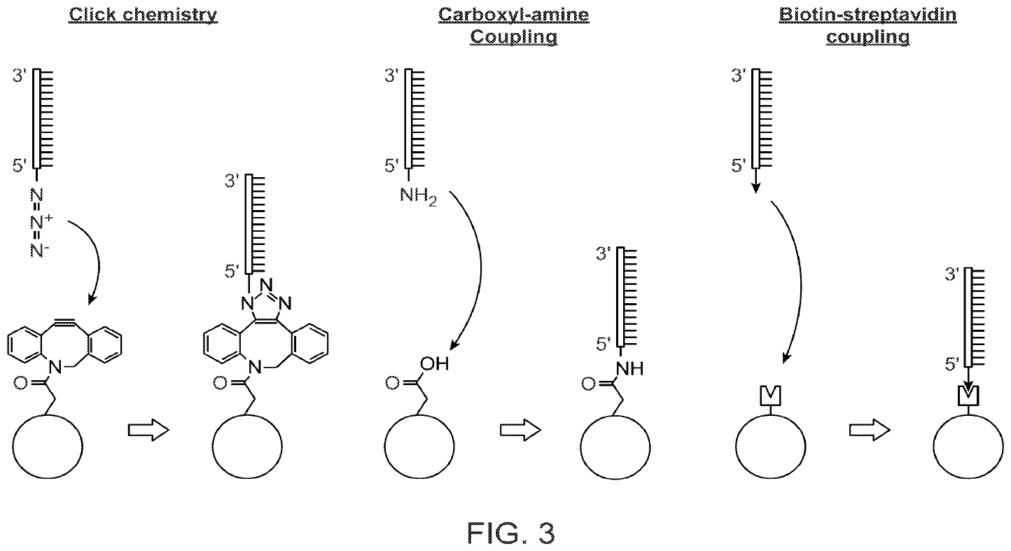

Various chemistries can be used to attach oligonucleotides to the solid support. In some embodiments, 5′ oligonucleotide modifications that are compatible with different types of beads are used. Representative examples of 5′ oligonucleotide/bead modifications include: Biotin/streptavidin, sulfhydryl/NHS ester, and Azide/DBCO (click chemistry). In some embodiments, a primal), amine is added to the oligonucleotide which allows reaction with NHS Esters on the solid support surface. In some embodiments, amino-modified oligonucleotides can be coupled to carboxylic acid modified solid supports via 5′ amino modification using 1-ethyl=3-(3-dimethylaminopropl)-carbodiimide hydrochloride (EDC) to form an amide bond. Representative methods for coupling oligonucleotides to solid supports are shown in FIG. 3. Methods for attaching oligonucleotides to solid supports are described in “Strategies for Attaching Oligonucleotides to Solid Supports” (Integrated DNA Technologies, 2014, v6).

### Barcodes

In some embodiments, the oligonucleotide attached to the solid support comprises an identification sequence, also referred to as a nucleic acid barcode, that can be used to identify the solid supports that bind mRNA from single cells. Examples of suitable barcodes are described in PCT/US2012/000221 (corresponding to US 2015/0133317) and PCT/US2014/072898 (corresponding to US 2015/0329891), which are incorporated by reference herein.

In some embodiments, the oligonucleotide attached to the solid support comprises two different or two distinct barcode sequences. In some embodiments, one (or a first) barcode sequence identifies the sample from which the mRNA transcripts were isolated. In some embodiments, the sample comprises one or more cells, or a single cell. Thus, in some embodiments, the first barcode is referred to as a “cell barcode.” In some embodiments, another (or a second) barcode sequence identifies the transcript isolated from a sample, such as a cell. Thus, in some embodiments, the second barcode is referred to an a “transcript barcode.”

In some embodiments, the barcode sequence comprises 8 to 32 nucleotides. In some embodiments, the first and/or second barcode sequence comprises 8 to 16 nucleotides. In some embodiments, the barcode sequence comprises 16 to 32 nucleotides.

In some embodiments, the solid support is linked to the oligonucleotide by a linker or spacer. In some embodiments, the linker or spacer comprises 5 or more nucleotides.

In some embodiments, the oligonucleotide attached to the solid support further comprises a poly-T sequence. In some embodiments, the poly-T sequence comprises 10-25 nucleotides.

In some embodiments, the oligonucleotide attached to the solid support comprises a linker or spacer of 5 or more nucleotides, a first or cell barcode sequence of 8 to 16 nucleotides, a second or transcript barcode sequence of 8 to 16 nucleotides, and a poly T sequence of 10-25 nucleotides.

### Libraries

Also provided are libraries of linked amplicons produced by the methods described herein. The libraries comprise physically linked amplicons produced by reverse transcription of mRNA in a first container, and amplification and linking of amplicons in a second container. In some embodiments, the linked amplicons are derived from the same cell, i.e., they are amplified from cDNA prepared by reverse transcription of mRNA from the same cell in a first container.

In some embodiments, the library comprises linked amplicons that encode IgG heavy and light chain sequences from B-cells. In some embodiments, the library comprises linked amplicons that encode IgG heavy and light chain sequences from a single or the same B-cell. In some embodiments, the library comprises linked amplicons that encode cognate pairs of IgG heavy and light chain sequences. The linker between the amplicons can include a linker for scFv antibody fragment expression or a constant region sequence for Fab antibody fragment expression.

In some embodiments, the library comprises linked amplicons that encode alpha and beta chains of the T cell receptor. In some embodiments, the library comprises linked amplicons that encode cognate pairs of alpha and beta chains of the T cell receptor. In some embodiments, the library comprises linked amplicons that encode alpha and beta chains of the T cell receptor from a single or the same T cell.

In some embodiments, expression (e.g., transcription and/or translation) of the amplified nucleic acid sequences is not desired. In these embodiments, the linker can be any stretch of nucleotides, e.g., 15-30 nucleotides in length, without significant secondary structure.

### Containers

In some embodiments, the first and/or second container is a tube, a well in a multiwell or microtiter plate, a well in a microwell or nanowell plate, a partition, a droplet or nanodroplet, or a microvesicle. In some embodiments, the first and/or second container is an aqueous droplet in an oil emulsion.

In some embodiments, the droplet has a diameter of about 2 micrometers to about 500 micrometers, or any value in between. For example, in some embodiments, the droplet has a diameter of about 2 to about 450 micrometers, about 2 to about 400 micrometers, about 2 to about 350 micrometers, about 2 to about 300 micrometers, about 2 to about 250 micrometers, about 2 to about 200 micrometers, about 2 to about 150 micrometers, about 2 to about 100 micrometers, about 2 to about 50 micrometers; about 2 to about 20 micrometers; about 5 to about 500 micrometers, about 5 to about 450 micrometers, about 5 to about 400 micrometers, about 5 to about 350 micrometers, about 5 to about 300 micrometers, about 5 to about 250 micrometers, about 5 to about 200 micrometers, about 5 to about 150 micrometers, about 5 to about 100 micrometers, about 5 to about 50 micrometers, about 5 to about 20 micrometers; about 10 to about 500 micrometers, about 10 to about 450 micrometers, about 10 to about 400 micrometers, about 10 to about 350 micrometers, about 10 to about 300 micrometers, about 10 to about 250 micrometers, about 10 to about 200 micrometers, about 10 to about 150 micrometers, about 10 to about 100 micrometers, about 10 to about 50 micrometers; or about 20 to about 500 micrometers, about 30 to about 500 micrometers, about 40 to about 500 micrometers, about 50 to about 500 micrometers, about 60 to about 500 micrometers, about 70 to about 500 micrometers, about 80 to about 500 micrometers, about 90 to about 500 micrometers, about 100 to about 500 micrometers, about 200 to about 500 micrometers, about 300 to about 500 micrometers, or about 400 to about 500 micrometers. In some embodiments, droplet has a diameter of about 2 micrometers to about 10 micrometers, for example, about 2 micrometers to about 5 micrometers.

In some embodiments, the first and second containers are aqueous droplets. In some embodiments, diameter of the first droplet is the same or similar to the diameter of the second droplet. In some embodiments, diameter of the first droplet is different from the diameter of the second droplet.

### Solid Supports

In some embodiments, the first container comprises one or more solid supports attached to an oligonucleotide comprising a sequence complementary to a portion of the mRNA molecules. In some embodiments, the mRNA molecules are attached to the oligonucleotide via binding to the complementary sequence. In some embodiments, the solid support is a bead, magnetic bead, agarose bead, or a particle. A bead or particle attached to an oligonucleotide comprising a sequence complementary to a portion of the mRNA molecules is sometimes referred to herein as a “capture bead.” While the term “bead” may be used to describe embodiments herein, it is understood that the term solid support can be used interchangeably with the term bead.

In some embodiments, the mRNA attached to the oligonucleotide is reverse transcribed into cDNA. In some embodiments, the CDNA is covalently linked to the solid supports.

In some embodiments, between 1 and 20 solid supports are present in the first container (e.g., 1, 2, 3, 4, 5, 6, 7, 8, 9, 10, 11, 12, 13, 14, 15, 16, 17, 18, 19 of 20 solid supports are present in the first container. In some embodiments, an average of 3, 4, 5, or 6 solid supports are present in the first container.

In some embodiments, the solid support is a spherical particle having a diameter of 1 to 20 micrometers, or a diameter of 5 to 10 micrometers, or an average diameter between 5 and 10 micrometers. In some embodiments, the solid support is a bead having a diameter of 1 to 20 micrometers, or a diameter of 5 to 10 micrometers, or an average diameter between 5 and 10 micrometers.

Methods for attaching or conjugating nucleic acids and oligonucleotides to solid supports are known in the art, and include amino oligo conjugation to solid supports comprising N-hydroxysuccinimide (NHS) ester ligands, where the oligonucleotide is modified with a primary amino group that reacts with N-hydroxysuccinimide (NHS) functional groups to form a stable amide linkage. Other examples of commonly used strategies include—but are not limited to—conjugating biotinylated oligo to streptavidin-functionalized solid supports, and conjugating thiolated oligo to gold solid supports or to maleimide-functionalized supports.

### Microfluidic Systems

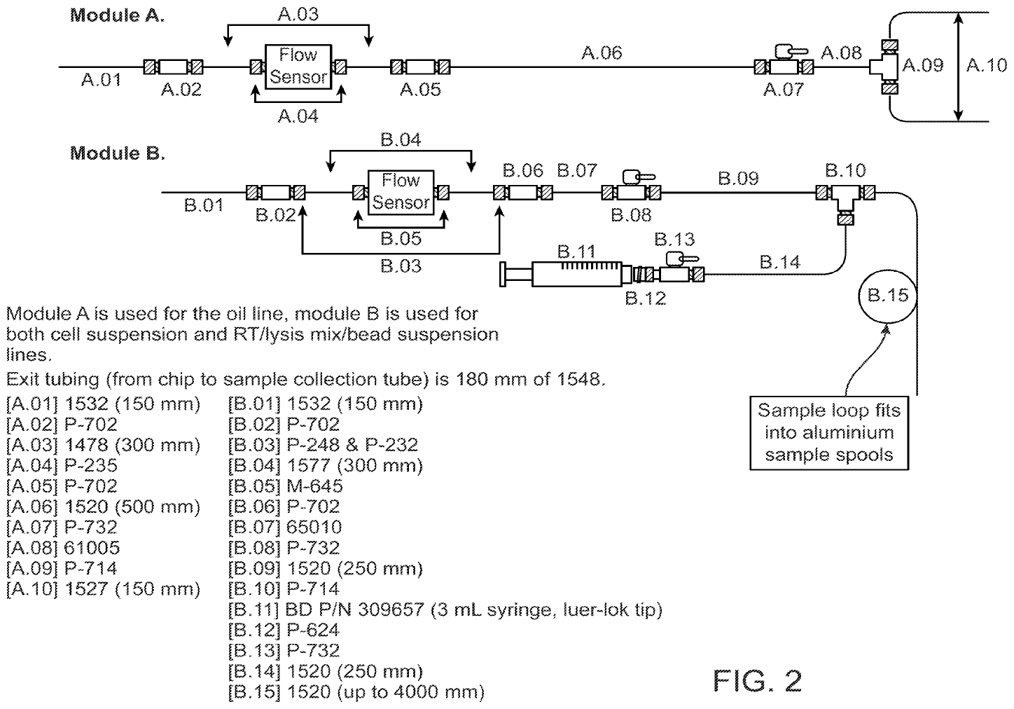

In some embodiments, a microfluidic system is used for producing aqueous-in-oil droplets for sequestering cells with mRNA capture beads and other molecular biology components needed to carry out cell lysis and the reverse transcription reaction. FIG. 2 shows a representative example of one system—a droplet device—which was described previously in U.S. patent application Ser. No. 14/586,857 (US 20150329891; now U.S. Pat. No. 9,580,736), which is incorporated by reference herein. The device joins aqueous streams of cell suspension, bead suspension, and cell lysis/RT mix at a junction with flow-focusing oil channels that break off aqueous droplets of near-uniform volume and at regular intervals. This monodisperse set of droplets are kept separate from one another by an oil phase that also includes a surfactant that stabilizes individual droplets and keeps them from joining or exchanging their contents to any significant degree. As such, each droplet comprises a container in which lysis and reverse transcription may proceed without influence from surrounding droplets. For a given droplet size, the average number of cells and barcoded beads per droplet can be adjusted by adjusting the concentration of those components in their respective aqueous input streams. There are a variety of choices for oil and surfactant systems to be used for droplet generation on the microfluidic device. In some embodiments, the oil phase comprises 2% fluorosurfactant (RAN Biotechnologies, Beverly, Mass.) in HFE-7500 fluorinated oil (3M, St. Paul, Minn.).

It will be understood by a person of ordinary skill in the art that the methods described herein could be used with any number of different oil/surfactant systems as long as they promote sufficient droplet stability and compatibility with the molecular biology that is involved. Typical oil systems include fluorinated oil, mineral oil, and silicone oil, and any of these could potentially be used at any of the emulsion steps.

### Samples

The methods described herein can be applied to biological samples comprising cells. The methods described herein can be used in any application involving the joining of multiple transcriptomic targets from any given population of single cells. The methods can be used with many different cell types from different biological tissues. The cells can be isolated from a mammal, including but not limited to mice, rats, companion animals such as cats and dogs, farm animals such as cows, pigs, and horses, and humans. In some embodiments, the cells are sorted into individual single cells. Individual single cells can be sorted, for example, using flow cytometry, fluorescence-activated cell sorting (FACS), magnetic-activated cell sorting (MACS) or panning. In some embodiments, single cells are added to a container described herein, such as an aqueous-in-oil droplet.

In some embodiments, the sample includes single immune cells, such as single B cells or single T cells (T lymphocytes). B-cells include, for example, activated B cells, blasting B cells, plasma cells, plasmablasts, memory B cells, B1 cells, B2 cells, marginal-zone B cells, and follicular B cells. T-cells (T lymphocytes) include, for example, cells that express T cell receptors. T cells include activated T cells, blasting T cells, Helper T cells (effector T cells or Th cells), cytotoxic T cells (CTLs), memory T cells, central memory T cells, effector memory T cells and regulatory T cells. In some embodiments, the sample includes natural killer T (NKT) cells.

In some embodiments, the B cell is an activated B cell that is about 8-20 μm in diameter, for example, about 8, 9, 10, 11, 12, 13, 14, 15, 16, 17, 18, 19, 20, or greater than 20 μm in diameter. In some aspects, the activated B cell is about 60, 70, 80, 90, 100, 120, 130, 140, 150, 200, 250, 300, 350, or greater than 350 μm² in area. In some aspects, the activated B cell is about 250, 268, 300, 400, 500, 600, 700, 800, 900, 1000, 2000, 3000, 4000, or greater than 4000 μm³ in volume. In some aspects, the activated B cell has a diameter of 10% or greater, 15% or greater, or 20% or greater in size than the median diameter of a control resting B cell. In some aspects, the activated B cell is capable of secreting immunoglobulin. In some aspects, the B cell has a forward scatter (FSC) greater than 1.2× of the FSC mean of resting B lymphocytes by flow cytometry. In some aspects, the B cell has a FSC mean between 0.7-1.15× of the FSC mean of human monocytes by flow cytometry. In some aspects, the B cell is a CD19 positive B cell, a CD38 positive B cell, a CD27 positive B cell, or a CD20 negative B cell. In some aspects, the B cell is a CD19+CD20−CD27+CD38hi B cell.

Individual B cells can be sorted by flow cytometry from blood, bulk peripheral blood mononuclear cells (PBMCs), bulk B cells, plasmablasts, plasma cells, memory B cells, or other B cell populations.

Methods for sorting B cells into single cells are described in US 2015/0133317. Briefly, blood can be collected in heparin tubes (Beckton Dickinson and Company, catalog #BD366664) or in CPT tubes (Beckton Dickinson and Company, catalog BD362761) tubes. In one representative method for processing heparin tubes, one milliliter of blood is transferred into a microfuge tube and spun down at 12,000 rpm for 3 minutes, plasma is collected and frozen at −80° C. (for later testing for antibody reactivities), the remainder of the blood can be layered over Ficoll and centrifuged in a Beckman Coulter Allegra X-15R benchtop centrifuge with a SX4750 Swinging Bucket Rotor at 800 g for heparin tubes for 20 min at room temperature, with minimal acceleration and without use of the brake, and the peripheral blood mononuclear cell (PBMC) layer is collected. Alternatively, CPT tubes can be directly centrifuged at 1,500 g for 20 min at room temperature, with minimal acceleration and without use of the brake, and the PMBC layer collected. The collected PBMCs can be washed twice with PBS before use.

### Isolation and Enrichment of Cells and Cell Subpopulations

Plasmablasts. For some samples, PBMCs can be enriched for plasmablasts by using a modified Plasma Cells Isolation Kit II (Miltenyi 130-093-628).

Memory B-cells. CD19+ microbeads (Miltenyi 130-050-301) and CD27+ microbeads (130-051-601) may be used to enrich for memory B-cells before cell sorting, to shorten sort times. Other enrichment methods, such as Memory B-cell isolation kit (Miltenyi 130-093-546), may also be used, provided that they enrich for CD19⁺CD27⁺ cells.

Total B-cells. CD19+ microbeads (Miltenyi 130-050-301) may be used to enrich for total B-cells before cell sorting, e.g., to shorten sort times. Other enrichment methods may also be used that enrich for CD19⁺ cells.

Other cell types. MACS enrichment of the desired cell population can shorten sort times. Other cell populations, including plasma cells, other B-cell populations and non-B-cell populations may also be enriched using MACS or other systems using the appropriate reagents. For example, total T-cells may be enriched using CD3+ microbeads, and effector T-cells and helper T-cells isolated using CD8+ and CD4+ microbeads, respectively. CD45RO microbeads may be used to isolate memory T-cells and, in conjunction with CD8+ or CD4+ beads, used to isolate memory effector or memory helper T-cells, respectively.

### Single-Cell Sorting

MACS enrichment is not required for sorting, but MACS enrichment for plasmablasts may be performed to shorten sort times. If PBMCs have undergone MACS enrichment, an aliquot of unenriched PBMCs (˜1 million cells) can also be analyzed in tandem, allowing the baseline plasmablast percentage in the sample to be determined. For sorting plasmablasts, cells can be stained with manufacturer-recommended volumes of CD3-V450 (BD 560365), IgA-FITC (AbD Serotec STAR142F), IgM-FITC (AbD Serotec STAR146F) or IgM-PE (AbD Serotec STAR146PE), CD20-PerCP-Cy5.5 (BD 340955), CD38-PE-Cy7 (BD 335808), CD19-APC (BD 340437) and CD27-APC-H7 (BD 560222) in 50 μL of FACS buffer (PBS or HBSS with 2% FBS) on ice for 20 minutes in the dark. Some cells may also be stained with IgG-PE (BD 555787), CD138-PE (eBioscience 12-1389-42), or HLA-DR-PE (BD 555812) together with IgM-FITC instead. For simultaneous sorting of plasmablasts, memory and naive B-cells, the following staining scheme can be used: IgD-FITC (Biolegend 348205), IgG-PE (BD 555787), CD20-PerCP-Cy5.5, CD38-PECy7, IgM-APC (BD 551062), CD27-APC-H7, IgA-biotin (AbD Serotec 205008) followed by Strepavidin-eFluor710 (eBioscience 49-4317-82) and CD19-BV421 (Biolegend 302233). Memory B-cells can be sorted either as CD19⁺CD27⁺IgG⁺ or CD19⁺CD20⁺IgG⁺, naive B-cells can be sorted as CD19⁺IgD⁺IgM⁺. IgA⁺ plasmablasts are defined as CD19⁺CD20⁻CD27⁺CD38⁺⁺IgA⁺IgM⁻. Other cell surface markers may also be used, as long as the B-cell or other cell population is phenotypically identifiable using cell surface markers, the population can be single-cell sorted. Cells can then be washed once with 2 mL of FACS buffer and resuspended at an appropriate volume for FACS. Cells can first be sorted on a BD Aria II into a 5 mL round bottom tube. Typically, purities of >80% are achieved from the first sort. For IgG⁺ plasmablasts, the gating (selection of cells) strategy can comprise sorting for the makers CD19⁺CD20⁻CD27⁺CD38⁺⁺IgA⁻IgM⁻. Sorted plates can be sealed with aluminum plate sealers (Axygen PCR-AS-600) and immediately frozen on dry ice and stored at −80° C.

Single-Cell Sorting Gating Strategies.

B-cells. For B-cells, the gating approach can comprise sorting for one or more of the following markers: IgM, IgG, IgA, IgD, CD19, or CD20. For total IgG⁺ B-cells, the gating approach can comprise sorting for IgG⁺. For total IgA⁺ B-cells, the gating approach can comprise sorting for IgA⁺. For total IgM⁺ B-cells, the gating approach can comprise sorting for IgM⁺.

Activated B cells. Activated B cells include B cells that have been stimulated through binding of their membrane antigen receptor to its cognate antigen and/or have received T cell help from T cells recognizing epitopes derived from the same macromolecular antigen. Activated B cells can be identified by a variety of properties including increased cell size (e.g. “blasting B cells”; see below), expression of cell surface marker or markers, expression of intracellular marker or markers, expression of transcription factor or factors, exiting the gap 0 (G0) phase of the cell cycle, progressing through the cell cycle, production of cytokines or other factors, and/or the down regulation of certain cell surface marker or markers, intracellular marker or markers, transcription factor or other factor. One method of identifying an activated B cell is to combine detection of a B cell marker such as CD19 or immunoglobulin with a marker of activation such as increased cell size or volume, the cell surface activation marker CD69, or progression through the cell cycle based on cell-permeable acridine orange DNA stain or another cell cycle analysis.

Blasting B cells. “Blasting B cells” are B cells that are activated and increased in size relative to resting B cells. Blasting B cells include the plasmablast population as well as other populations of activated B cells, and blasting B cells are physically larger in size than resting B cells. Blasting B cells can be single-cell sorted using several different approaches, including gating (selection) of B cells based on their physically being larger based on cell diameter, cell volume, electrical impedance, FSC, the integral (area) of a FSC pulse (FSC-A), FSC height (FSC-H), forward scatter pulse width (FCS-W), side scatter (SSC), side scatter pulse area (SSC-A), side scatter height (SSC-H), side scatter width (SSC-W), autofluorescence and/or other measures of cell size.

In flow cytometry, forward scatter (FSC) is measured using a light beam in line with the stream of cells and provides information regarding the proportional size and diameter of each cell. Using FSC one can select B cells with FSC greater than the median FSC of resting B cell, for example an FSC-A or FSC-H 5% greater than resting B cells, 10% greater than resting B cells, 15% greater than resting B cells, 20% greater than resting B cells, 30% greater than resting B cells, 40% greater than resting B cells, 50% greater than resting B cells, 60% greater than resting B cells. By analyzing calibration beads of specific sizes, one can use FSC to determine the relative size of B cells relative to the calibration beads. By doing so, one can specifically gate on and thereby select B cells that possess diameters of about 8 um, >8 um, >9 um, >10 um, >11 um, >12 um, >13 um, >14 um, >15 um, >16 um, >17 um, >18 um, >19 um, or >20 um.

Another measurement of cell size is cell volume. The “gold standard” for cell volume uses the Coulter principle which is based on an electronic measurement (Tzur et al, PLoS ONE, 6(1): e16053. doi:10.1371/journal.pone.0016053, 2011). Although the method of sorting by droplet charging and deflection was first used in a device that measured cell volume by impedance, the currently available flow cytometers make only optical measurements. FSC measurements, specifically the FSC-A (FSC integral area) are commonly used to assess cell size, although FSC measurements can be influenced by the refractive index differences between particles and fluid (Tzur et al, PLoS ONE, 6(1): e16053. doi:10.1371/journal.pone.0016053, 2011). Some have shown that volume estimation can be improved by combining optical parameters, including FSC-W, SSC and 450/50-A auto fluorescence (Tzur et al, PLoS ONE, 6(1): e16053. doi:10.1371/journal.pone.0016053, 2011).

For example, selection of activated B cells based on increased size can be achieved through identifying B cells using a marker such as CD19 and assessing size through FSC or FSC-A. Other B cell markers and/or parameters for assessment of size are described herein.

Plasmablasts. For isolation of plasmablasts, the gating approach can comprise sorting for CD19⁺CD38⁺⁺ B-cells. For isolation of IgG⁺ plasmablasts, the gating approach can comprise sorting for CD19⁺CD38⁺⁺IgA⁻IgM⁻ B-cells. For isolation of IgA+ plasmablasts, the gating approach can comprise sorting for CD19⁺CD38⁺⁺IgA⁺ B-cells. For isolation of IgM+ plasmablasts, the gating approach can comprise sorting for CD19⁺CD38⁺⁺IgM⁺ B-cells. In addition, other gating strategies can be used to isolate a sufficient number of plasmablasts to carry out the methods described herein. Plasmablasts can also be isolated using the following marker expression patterns CD19^{low/+}, CD20^{low/-}, CD27⁺ and CD38⁺⁺. Although use of all these markers generally results in the purest plasmablast population from single cell sorting, not all of the above markers need to be used. For example, plasmablasts may also be isolated using the following gating strategies: forward scatter high (FSC^{hi}) for larger cells, FSC^{hi}CD19^{lo} cells, FSC^{hi} and CD27⁺, CD38⁺⁺, or CD20⁻ cells. Combination of any of these markers or other markers found to be able to distinguish plasmablasts from other B-cells will generally increase the purity of sorted plasmablasts, however any one of the above markers alone (including FSC^{hi}) can distinguish plasmablasts from other B-cells, albeit with a lower purity.

Memory B-cells. For IgG⁺ memory B-cells, the gating approach can comprise sorting for CD19⁺CD27⁺IgG⁺ or CD19⁺CD20⁺IgG⁺. For IgA⁺ memory B-cells, the gating strategy can comprise CD19⁺CD27⁺IgA⁺ or CD19⁺CD20⁺IgA⁺. For IgM⁺ memory B-cells, the gating strategy can comprise CD19⁺CD27⁺IgM⁺ or CD19⁺CD20⁺IgM⁺.

Other cell types. As long as the B-cell, T-cell, or other cell population is phenotypically identifiable using cell markers, it can be single-cell sorted. For example, T-cells can be identified as CD3⁺ or TCR⁺, naïve T-cells identified as CD3⁺CD45RA⁺, memory T-cells identified as CD3⁺CD45RO⁺. Effector and helper T-cells can be identified as CD3⁺CD8⁺ and CD3⁺CD4⁺, respectively. Cell populations can be further subdivided by using combinations of markers, such as CD3⁺CD4⁺CD45RO⁺ for memory helper T-cells.

### EXAMPLES

The examples are offered for illustrative purposes only, and are not intended to limit the scope of any embodiment of the present invention in any way. Efforts have been made to ensure accuracy with respect to numbers used (e.g., amounts, temperatures, etc.), but some experimental error and deviation should be allowed for.

### Example 1

This example describes a representative embodiment of the methods described herein.

In the first droplet (droplet 1), cells are individually encapsulated, one (1) cell per droplet, and the droplets typically contain greater than 10 mRNA capture beads/droplet. The cell in the droplet is lysed, and a reverse transcription reaction occurs in droplet 1. The RT reaction comprises temporarily hybridizing poly-A RNA to oligo-dT conjugated beads. cDNA synthesis then occurs by primer extension of the oligo dT primer, resulting in cDNA that is covalently-linked to the capture beads in droplet 1. The RNA hybridized to the cDNA is destroyed by enzymatic digestion in droplet 1, and therefore RNA is not isolated from droplet 1. Droplet 1 is broken, and the beads are washed. The washed beads isolated from droplet 1 do not contain appreciable amounts of cellular material, including RNA, from the lysed cells.

The isolated beads comprising the covalently-linked cDNA are then incorporated into a second droplet (droplet 2) at a concentration of 1 or less beads/droplet. A PCR reaction is performed to link the heavy and light chain cDNAs, and release the linked heavy and light chain cDNAs from beads.

### Example 2

This example describes a representative embodiment of the methods described herein.

Reverse Transcription (RT) in Droplet 1

Materials:

RT reaction reagents: Invitrogen SuperScript IV RT (ref #18090050), dNTPs, 1M DTT, BSA, Ribolock, Tween-20, NEB RNaseH (Cat #M0523S); Oligo d(T) 25 beads (NEB, Cat #S1419S); 1× Bead Wash Buffer (BWB): 5 mM Tris-HCl (pH 7.5), 0.5 mM EDTA, 1M NaCl; PBS; Optiprep (Sigma, Cat #D1556); FC40 5% Ran; Plastics: Striptubes, 1.5 mL microcentrifuge tubes; Mushroom magnet; ZeroStat Milty anti-static gun.

RT reaction mix.

1. Make RT master mix:

| SSIV RT | Stock Conc | | Final Conc | | 300 uL |
|---|---|---|---|---|---|
| SSIV RT buffer | 5 | x | 2 | x | 120.0 |
| dNTPs | 10 | mM | 1 | mM | 30.0 |
| DTT | 1000 | mM | 10 | mM | 3.0 |
| BSA | 20 | mg/mL | 1 | mg/mL | 15.0 |
| Ribolock | 40 | U/uL | 0.5 | U/uL | 3.8 |
| SSIV RT enzyme | 200 | U/uL | 20 | U/uL | 30.0 |
| RNaseH | 5 | U/uL | 0.05 | U/uL | 3.0 |
| Tween-20 | 20% | | 1% | | 15.0 |
| water | | | | | 80.3 |

2. Thaw vials of FACS sorted cells.
3. Resuspend cells in PBS with 20% optiprep, at 1×10\hat{ }6 cells/mL (or 1000 cells/uL), concentration and divide into 100 uL aliquots.
4. Take 45 uL (enough for 1×10⁶ cells in 1 mL) of OdT beads to a 1.5 mL tube and place the tube on magnet. Aspirate storage buffer and wash once with 500 uL of BWB. Place on magnet again and resuspend in 300 uL of PBS. Calculate the volume of beads needed for the run: e.g. for 300 thousand cells, 300 uL/1,000,000 cells*300,000 cells=90 uL.
5. Resuspend OdT beads in the correct volume of RT reaction master mix (“mmix”; same volume as cells) and divide into 100 uL aliquots.
6. Proceed to running samples through droplet machine.

Droplet 1 Formation:

One example of a functional droplet device is shown in FIG. 2. All part numbers listed refer to IDEX Health & Science (Oak Harbor, Wash.) parts unless otherwise specified. A.01 and B.01 connect to pressure pumps where A.01 reservoirs are filled with HFE-7500 fluorinated oil with 2% RAN fluorosurfactant and the B.01 reservoir is filled with water. B.11 is connected to a syringe pump.

7. Separately load the cell suspension and the RT/lysis/bead suspension in two reservoirs of the droplet device using syringe pumps to draw the sample into the sample loops. Maintain a 1 cm air gap between the pushing fluids and the samples in B.15.
8. A.10 and B.15 are connected to a 100 um etch depth fluorinated 2R chip (Dolomite part number 3200510)
9. Run the sample at flow rates of 15 uL/min for each aqueous line (B.15) and 180 uL/min for the oil line (A.10).
10. The emulsion is collected in a 15 mL conical tube that is kept on ice.

Droplet 1 Cleanup and RT/RNaseH Incubation:

11. Set up two 1.5 mL tubes with 500 uL of 5% Ran FC40 in each tube.
12. A wide bore tip was used for handling emulsions, and an anti-static gun (ZeroStat Milty) was used on the tip to prevent shearing of emulsion.
13. Collect emulsion from 15 mL Falcon and aspirate into one of the tubes with 5% Ran. Aspirate some 5% Ran from the bottom of the tube and gently wash emulsion with it. Repeat.
14. Collect emulsion from the first wash tube to the other and wash again.
15. Collect washed emulsion to a striptube, 90 uL/well, and place on thermocycler.
16. Run RT+RNaseH thermocycling program: 55 C 20 min, 65 C 10 min, 80 C 10 min.

Droplet 2/PCR1:

Materials Needed:

Lithium Lysis Buffer: 100 mM Tris (pH 7.5), 500 mM LiCl, 10 mM EDTA, 1% (w/v) lithium dodecyl sulfate, 5 mM DTT.

Wash Buffer 1: 100 mM Tris (pH 7.5), 500 mM LiCl, 1 mM EDTA.

Wash Buffer 2: 20 mM Tris (pH 7.5), 3 mM MgCl, 50 mM KCl.

20 mL Oil mixture: 19010 uL mineral oil, 900 uL Span80, 80 uL Tween-80, 10 uL Triton X-100.

20% PFO.

KOD Xtreme Hot Start DNA Polymerase (Millipore, Cat #71975).

Forward and reverse primers for target amplicons, such as V and J gene primers for the heavy and light chain variable regions.

IKA dispersing tube+emulsion-dispersing apparatus

Zymo DNA Clean and Concentrate kit.

Dynabeads MyOne C1 Streptavidin Beads (Cat #65001, 65002).

2× Bead Wash Buffer (BWB): 10 mM Tris-HCl (pH 7.5), 1 mM EDTA, 2M NaCl.

Plates/tubes: 98-well plates, 1.5 mL tubes.

RT product wash and setting up PCR1:

1. Make fresh lithium lysis buffer daily. Wash buffers 1 and 2 should be made fresh every 3-6 months.
2. Make fresh oil mixture and place on ice.
3. Make PCR1 reaction mix:

| | | | | | Primer Conc |
|---|---|---|---|---|---|
| | | | | | 8, total |
| KOD Xtreme | Stock conc | | Final conc | | vol 150 uL |
| 2x Xtreme Buffer | 2 | x | 1 | x | 75.0 |
| dNTPs | 10 | mM | 0.4 | mM | 6.0 |
| Fwd Adaptor #5459 | 10 | uM | 0.4 | uM | 6.0 |
| Outside F #5462 | 10 | uM | 0.3 | uM | 4.5 |
| Inside R #5463 | 10 | uM | 0.08 | uM | 1.2 |
| Inside F #5464 | 10 | uM | 0.075 | uM | 1.1 |
| Outside R #5465 | 1 | uM | 0.01 | uM | 1.5 |
| Rev Adaptor #5460 | 10 | uM | 0.4 | uM | 6.0 |
| BSA (20 mg/mL) | 20 | mg/mL | 0.5 | mg/mL | 3.8 |
| Ribolock (40 U/uL) | 40 | U/ul | 0.24 | U/ul | 0.9 |
| KOD Xtreme Hot Start | 1 | U/ul | 0.02 | U/ul | 3.0 |
| polymerase | | | | | |
| water | | | | | 41.0 |

4. Collect reverse transcribed emulsion from striptubes to a new 1.5 mL and add equal volume of 20% PFO (1:1 ratio) to break emulsion.
5. Vortex samples and centrifuge for 2 min at 2000×g.
6. Remove oil layer (bottom) and measure aqueous layer (top) and transfer the supernatant to a new tube.
7. Place sample on magnet.
8. Be quick with all wash steps and avoid mixing by pipetting to prevent beads becoming sticky and getting stuck inside the pipette tip.
9. Aspirate supernatant and add 100 uL of Wash Buffer 1. Mix by pipetting (exception) and immediately move to a new tube. Place on magnet.
10. Aspirate supernatant and add 100 uL of lithium lysis buffer. Do not take the tube off the magnet. Mix by turning the tube: the beads should go from one side of the tube to the other. If the beads seem sticky, tap on the side of the tube to help them dislodge.
11. Aspirate supernatant and add 100 uL of Wash Buffer 1 and immediately aspirate.
12. Add 100 uL Wash Buffer 1 again and wash by keeping the tube on magnet but turning it (like in step 10).
13. Aspirate supernatant and add 100 uL Wash Buffer 2 and wash by keeping the tube on magnet but turning it (like in step 10).
14. Place on magnet, aspirate supernatant, and add 200 uL of PCR1 mmix to the beads. Let sit on ice for a couple of minutes. Resuspend well by pipetting up and down.
15. Add more PCR1 mmix to sample, up to 2.8 mL
16. Fill IKA dispersing tube with 9 mL of cold oil mixture and attach the tube to the emulsion-dispersing apparatus. Set the unit to 600 rpm and dispense PCR1 mmix with beads to the oil dropwise using the electronic dispenser. Each sample takes 5 min to run and make emulsion.
17. Use a repeater pipette with a wide bore or cut-off tip and transfer 100 uL of the emulsion to each well of a 96-well plate. Each sample requires two plates.
18. Place plates on thermocyclers and run KOD xtreme thermocycling program named “KOD xtreme phage PCR1”: 94 C 2 min; 35 cycles: 94 C 30 s, 60 C 30 s, 68 C, 45 s; 68 C 5 min.

Post-PCR1 Processing of Samples:

19. Make hydrated ether by mixing water and diethyl ether 1:10 (1 mL water and 9 mL diethyl ether) in a fume hood. Vortex and place the mixture on ice and let settle for about 5 min. Only use the top layer.
20. Collect PCR1 emulsion from plates to 1.5 mL tubes, no more than 1 mL/tube.
21. Centrifuge tubes 10 min at 10,000×g.
22. After centrifuging, aspirate the top oil layer and any coalesced material on the bottom.
23. Estimate the volume of emulsion in the tube and add hydrated ether in 1:1 ratio (use fume hood).
24. Continue working in the fume hood until done with first Zymo cleanup.
25. Vortex well and centrifuge again 10 min at 10,000×g.
26. Aspirate the aqueous layer (bottom) to a new 1.5 mL tube. Measure the volume.
27. Zymo clean sample with 3× Binding Buffer. Follow Zymo SOP and elute in 20 uL (2×10 uL) of 10 mM Tris.

PCR2

Materials:

Q5 (NEB Cat #M0493S/L).

Agarose, SYBRSafe gel dye, TBE buffer

Qiagen MinElute Gel Extraction kit (Cat #28006)

10 mM Tris

Plastics: 1.5 mL tubes, striptubes or 96-well plates

Setting Up PCR2 Reactions:

Set up test PCR2 reactions to determine the right cycle number for each sample: Prepare master mix (25 uL total/cycle), use primers Fab-K/L-F-R2-v1 (pool) and #1032, and use 1 uL of PCR1 product as template.

Run 13, 17, 21, 25 cycles. Use thermocycling program: 98° C. 30 s; 13/17/21/25 cycles: 98° C. 15 s, 62° C. 30 s, 72° C. 45 s; 72° C. 5 min.

Once PCR is done, run 5 uL of product on gel.

Run actual PCR2 to make products ready for library preparation for sequencing: prepare master mix, and use 2 uL of PCR1 product as template.

After PCR is done, load the whole sample (50 uL) on gel.

Gel extract samples by using Qiagen MinElute Gel extraction kit. Elute in 20 uL of 10 mM Tris.

Post PCR2:

Ligation of adaptors for NGS sequencing can be done using standard sequencing kits and methods. See Rajan et al. (2018), “Recombinant human B cell repertoires enable screening for rare, specific, and natively paired antibodies.” Communications Biology, 1(1), 5, and McDaniel et al. (2016), “Ultra-high-throughput sequencing of the immune receptor repertoire from millions of lymphocytes.” Nature Protocols, 11(3), 429-442, for examples.

Mastermixes:

| SSIV RT mmix | Stock Conc | | Final Conc | | 300 uL |
|---|---|---|---|---|---|
| SSIV RT buffer | 5 | x | 2 | x | 120.0 |
| dNTPs | 10 | mM | 1 | mM | 30.0 |
| DTT | 1000 | mM | 10 | mM | 3.0 |
| BSA | 20 | mg/mL | 1 | mg/mL | 15.0 |
| Ribolock | 40 | U/uL | 0.5 | U/uL | 3.8 |
| SSIV RT enzyme | 200 | U/uL | 20 | U/uL | 30.0 |
| RNaseH | 5 | U/uL | 0.05 | U/uL | 3.0 |
| Tween-20 | 20% | | 1% | | 15.0 |
| water | | | | | 80.3 |

Lithium lysis buffer (5 mL)

| | Stock | Final | |
|---|---|---|---|
| | [C] | [C] | ul |
| Tris (pH 7.5) | 1M | 100 mM | 500 |
| LiCl | 8M | 500 mM | 312.5 |
| EDTA | 0.5M | 10 mM | 100 |
| Lithium dodecyl sulfate | | 1% w/v | 0.05 g |
| DTT | 1M | 5 mM | 25 |
| H2O | | | 4062.5 |

| | Stock | Final | 10 |
|---|---|---|---|
| Wash buffer 1 | [C] | [C] | mL |
| Tris (pH 7.5) | 1M | 100 mM | 1000 |
| LiCl | 8M | 500 mM | 625 |
| EDTA | 0.5M | 1 mM | 20 |
| H2O | | | 8355 |

| | Stock | | 10 |
|---|---|---|---|
| Wash buffer 2 | [C] | Final [C] | mL |
| Tris (pH 7.5) | 1M | 100 mM | 1000.0 |
| MgCl | 200 mM | 3 mM | 150.0 |
| KCl | 3M | 50 mM | 166.7 |
| H2O | | | 8683.3 |

| KOD Xtreme | Stock | | Final | | Primer Cone 8, |
|---|---|---|---|---|---|
| PCR1 mmix | conc | | conc | | total vol 150 uL |
| 2x Xtreme Buffer | 2 | x | 1 | x | 75.0 |
| dNTPs | 10 | mM | 0.4 | mM | 6.0 |
| Fwd Adaptor | 10 | uM | 0.4 | uM | 6.0 |
| Outside F | 10 | uM | 0.3 | uM | 4.5 |
| Inside R (Red = 1 uM) | 10 | uM | 0.08 | uM | 1.2 |
| Inside F (Red = 1 uM) | 10 | uM | 0.075 | uM | 1.1 |
| Outside R | 1 | uM | 0.01 | uM | 1.5 |
| Rev Adaptor | 10 | uM | 0.4 | uM | 6.0 |
| BSA (20 mg/mL) | 20 | mg/mL | 0.5 | mg/mL | 3.8 |
| Ribolock (40 U/uL) | 40 | U/ul | 0.24 | U/ul | 0.9 |
| KOD Xtreme Hot | 1 | U/ul | 0.02 | U/ul | 3.0 |
| Start polymerase | | | | | |
| water | | | | | 41.0 |

| 2x BWB | Stock conc | | Final conc | | 5 mL | |
|---|---|---|---|---|---|---|
| Tris-HCl | 1000 | mM | 10 | mM | 50 | uL |
| (PH7.5) | | | | | | |
| EDTA | 500 | mM | 1 | mM | 10 | uL |
| NaCl | 5000 | mM | 2000 | mM | 2000 | uL |
| H2O | | | | | 2940 | uL |

| PCR2 | Stock conc | | Final conc | | Vol (ul)/rxn |
|---|---|---|---|---|---|
| Nuclease-Free Water | | | | | 34 |
| 5X Q5 Reaction Buffer | 5 | x | 1 | x | 10 |
| 10 mM dNTPs | 10 | mM | 0.2 | mM | 1 |
| 10 uM Primer Fw, | 10 | uM | 0.25 | uM | 1.25 |
| Fab-K/L-F-R2-v1 | | | | | |
| 10 uM Primer Rv, #1032 | 10 | uM | 0.25 | uM | 1.25 |
| Template DNA | | | | | 2 |
| Q5 Hot Start DNA | | | | | 0.5 |
| Polymerase | | | | | |
| Total | | | | | 50 |

It will be understood by a person of ordinary skill in the art that a wide variety of other components might be used to serve the same function. Alternative components could be used as the lysis agent, RT enzyme, RNase, primer sequences, and DNA polymerase, among other components.

### Example 3

This example provides experimental data showing that performing the reverse transcription step in a separate container from the amplification and linking step improves native pairing of amplicons.

Experiments were performed to test the percent of correctly paired amplicons in a library when reverse transcription (RT) of mRNA from single cells was performed in the first container (droplet 1), between the first and second containers (between (B/w) droplets 1 and 2), or in the second container (droplet 2).

Methods:

Conditions Tested:

(i) RT in droplet1: followed standard procedure (v2.1 droplet phage display).

(ii) RT after droplet1 and wash: followed standard procedure for making droplet1, but instead of following with RT incubation broke the emulsion with 20% PFO and washed beads according to standard procedure (post-droplet1 wash steps). Performed RT on washed beads (standard conditions). After incubation, placed samples on magnet and washed beads again according to standard procedure (post-droplet1 wash steps). Resuspended washed beads in KOD Xtreme™ (EMD Millipore) reaction mix for PCR1 and continued to droplet2 step on DT-20. After PCR1 followed standard procedure.

(iii) RT in droplet2 (RT-PCR kit, SSIV and Titan):): followed standard procedure for making droplet1, but instead of following with RT incubation broke the emulsion with 20% PFO and washed beads according to standard procedure (post-droplet1 wash steps). Resuspended washed beads in RT-PCR reaction mix (either SuperScriptIV, Titan, or Quanta) and used DT-20 to make droplets for RT incubation and PCR1 cycling (followed each reaction mixes recommended cycling conditions). After PCR1 followed standard procedure.

Results: As shown in the Table below, reverse transcription in droplet 1 increased the percentage of amplicons that are correctly paired and linked to another amplicon from the same cell. % DNA native refers to the fraction of heavy chain amplicons that were correctly paired with the native light chain amplicon in the library. Purity refers to the fraction of the library that is natively paired versus non-natively paired. As can be seen in the Table, 65-77% of amplicons in the library were correctly paired when RT was performed in droplet 1, whereas 9-32% were correctly paired when RT was performed in between droplets 1 and 2, and 23-40% were correctly paired when RT was performed in droplet 2.

Unique CDRH3 was used to determine total recovery of linked amplicons in the library. RT in droplet 1 decreased total recovery.

| | | Droplet | B/w Droplet | Droplet |
|---|---|---|---|---|
| | | 1 | 1 and 2 | 2 |
| Repeat 1 | Unique CDRH3 | 867 | 2695 | 2427 |
| | % DNA native** | 0.777 | 0.324 | 0.233 |
| Repeat 2 | Unique CDRH3 | 1215 | 2282 | 2699 |
| | % DNA native** | 0.654 | 0.0934 | 0.401 |

The data provided above demonstrates that performing the RT step in the first container increases the native pairing of amplicons in a library produced by the methods described herein.

### Example 4

This example describes the use of a photocleavable linker between the bead and capture oligonucleotide increase the yield and purity of the amplified product. The method of this example is similar to that described in Example 1, with changes detailed below.

In this example, a custom mRNA capture bead is prepared by conjugating oligodT ssDNAs to beads. A photocleavable linker, such as the nitrobenzyl linker offered by IDT (modification code ‘/iSpPC/’) is positioned between the oligodT capture/priming sequence and the bead surface. To test for successful photocleavage and release of ssDNA from the bead, a suspension of beads are exposed to 365 nm UV light for six minutes. The suspension is then centrifuged to pellet the beads, and the supernatant is assayed by Qubit (Thermo Fisher Cat. No. Q10212) to determine the quantity of ssDNA that is released from the beads.

Reagents: DBCO-modified 10 μm diameter polystyrene beads (Creative Diagnostics Cat. No. DNM-M006). Azide and photocleavable linker-modified mRNA capture oligo (IDT):

(SEQ ID NO: 1)

5′ - Azide-iSpPC-TTTTTTTTTTTTTTTTTTTTTTTTT - 3′.

Droplet generation oil for EvaGreen (BioRad Cat. No. 1864112).

Methods:

The stock of mRNA capture beads were prepared as follows:
1. Wash 10 million DBCO beads 5× in 500 uL of 0.1×PBS+0.001% Tween-20
2. Transfer 8 million beads into a 0.5 mL PCR tube
3. Spin down, aspirate off supernatant
4. Resuspend the beads in the following reaction mix:

[stock]

| PBS | 10 | X | 22 | uL |
|---|---|---|---|---|
| EDTA | 100 | mM | 1.1 | uL |
| NaCl | 5000 | mM | 4.4 | uL |
| Tween-20 | 0.1% | | 2.2 | uL |
| Azide mRNA capture | | | | |
| oligo | 100 | uM | 2.2 | uL |
| Water | | | 188.1 | uL |

5. Vortex the tube, wrap in aluminum foil, place on a rotator or shaker incubating at 72 C for one hour
6. After incubation, transfer from the coupling tube to a separate 0.5 mL PCR tube
7. Spin down, aspirate supernatant, resuspend in 0.5 mL of 0.1×PBS+0.001% Tween-20
8. Wash 3× in 0.5 mL of 0.1× TE+0.001% Tween-20
9. Perform photocleavage/Qubit assay as described above to determine conjugation yield and test successful release of oligodT from the bead surface

The same procedure is followed as in Example 1, with the exceptions that Step 4 of paragraph [0126] (‘RT reaction mix”) uses 5 million custom beads rather than 45 uL of bead stock, and that Steps 15 & 16 of paragraph [0144] (“RT product wash and setting up PCR1”) were replaced by the following steps:

1. Add more PCR1 mmix to sample, up to 500 uL
2. Using the same droplet device as before with the A.01 reservoir now filled with BioRad droplet generation oil for EvaGreen and the B.01 reservoirs filled with water. B.11 is connected to a syringe pump.
3. Separately load one half (250 uL) of the bead/PCR mix suspension in each of the two reservoirs of the droplet device using syringe pumps to draw the sample into the sample loops. Maintain a 1 cm air gap between the pushing fluids and the samples in B.15.
4. A.10 and B.15 are connected to a 100 um etch depth fluorinated 2R chip (Dolomite part number 3200510)
5. Run the sample at flow rates of 12 uL/min for each aqueous line (B.15) and 180 uL/min for the oil line (A.10).
6. The emulsion is collected in a 15 mL conical tube that is kept on ice.
7. Following production of the emulsion and prior to aliquoting the emulsion into a PCR plate, expose the emulsion to 365 nm UV light for 6 minutes, rotating the tube so that all parts of the emulsion are evenly exposed.

By incorporating the UV cleavage step the inventors observed that the proportion of unique heavy chain sequences that were confidently paired with a light chain increased from 71.1% to 80.6%.

This example demonstrates that the both the number of heavy and light chains pairs recovered and the proportion of natively-paired heavy and light chains can be increased using the methods described above.

### Informal Sequence Listing:

SEQ ID NO: 1:

5′ - Azide-iSpPC-TTTTTTTTTTTTTTTTTTTTTTTTT - 3′.

It is understood that the examples and embodiments described herein are for illustrative purposes only and that various modifications may be suggested to persons skilled in the art after reviewing this disclosure, which are to be included within the scope of the appended claims. All publications, sequence accession numbers, patents, and patent applications cited herein are hereby incorporated by reference in their entirety for all purposes.

What is claimed is:

1. A method of producing two or more linked nucleic acid molecules from a single cell, comprising:
(i) isolating a single cell in a first container, and lysing the single cell to release mRNA molecules;
(ii) reverse transcribing the mRNA molecules to produce cDNA molecules in the first container; and
(iii) linking the cDNA molecules derived from the single cell in step (ii) in a second container,
thereby producing linked nucleic acid molecules.

2. The method of claim 1, wherein the first container comprises one or more solid supports attached to an oligonucleotide comprising a sequence complementary to a portion of the mRNA molecules.

3. The method of claim 2, wherein the mRNA molecules are attached to the oligonucleotide via binding to the complementary sequence.

4. The method of claim 3, wherein the reverse transcribing comprises extending the oligonucleotide with a reverse transcriptase to produce the cDNA molecules.

5. The method of claim 4, wherein the cDNA molecules from step (ii) are covalently linked to the solid supports.

6. The method of claim 5, wherein each of the one or more solid supports is isolated in a different second container prior to step (iii).

7. The method of claim 2, wherein the oligonucleotide is attached to the solid support by a linker.

8. The method of claim 7, wherein the linker is located between a surface of the solid support and the sequence complementary to a portion of the mRNA molecules.

9. The method of claim 7, wherein the linker is a photocleavable linker.

10. The method of claim 9, wherein the cDNA molecules are released from the solid support by exposing the photocleavable linker to light in the second container.

11. The method of claim 2, wherein 1 to 20 solid supports are present in the first container.

12. The method of claim 2, wherein an average of 3 to 5 solid supports are present in the first container.

13. The method of claim 2, wherein an average of 15 solid supports are present in the first container.

14. The method of claim 2, wherein the solid support is a bead or particle.

15. The method of claim 2, wherein the solid support is a spherical particle having a diameter of 1 to 20 micrometers.

16. The method of claim 2, wherein the solid support has an average diameter between 5 and 10 micrometers.

17. The method of claim 2, wherein linking the cDNA molecules in step (iii) comprises amplifying and linking the cDNA molecules by overlap extension PCR.

18. The method of claim 17, wherein the cDNA molecules are released from the solid support in the second container prior to step (iii).

19. The method of claim 17, wherein the overlap extension PCR comprises amplifying the cDNA molecules using one or more internal primers comprising a biotin tag.

20. The method of claim 19, wherein cDNA molecules comprising the biotin tag are removed after step (iii).

21. The method of claim 17, wherein the overlap extension PCR comprises amplifying the cDNA molecules using one or more external primers chemically modified to resist nuclease degradation.

22. The method of claim 21, wherein the one or more external primers are chemically modified to include phosphorothioate bonds.

23. The method of claim 22, wherein the cDNA molecules are contacted with a 5′-exonuclease after step (iii).

24. The method of claim 1, wherein the single cell is an immune system cell.

25. The method of claim 1, wherein the single cell is a B cell, a memory B cell, an activated B cell, a blasting B cell, a plasma cell, or a plasmablast.

26. The method of claim 25, wherein the mRNA molecules encode a heavy chain variable region and a light chain variable region.

27. The method of claim 25, wherein the cDNA molecules encode a cognate pair of heavy and light chain variable regions.

28. The method of claim 1, wherein the single cell is a T cell.

29. The method of claim 1, wherein the single cell is a natural killer T (NKT) cell.

30. The method of claim 27, wherein the cDNA molecules encode a cognate pair of T cell receptor alpha and beta chains.

31. The method of claim 1, wherein the first or second container comprises a partition, an aqueous droplet in an emulsion, a microvesicle, a tube, or a multiwell plate.

32. The method of claim 31, wherein the droplet is 2 to 500 micrometers in diameter.

33. The method of claim 1, further comprising digesting the mRNA following step (ii).

34. The method of claim 33, wherein the mRNA is digested in the first container, or between steps (ii) and (iii).

35. A method for producing a library of linked nucleic acid molecules, comprising:
a) isolating a plurality of single cells in a plurality of first containers, where the first containers comprise a single cell;
b) lysing the single cells to release mRNA molecules in the first container;
c) reverse transcribing the mRNA molecules to produce cDNA molecules derived from single cells in the first container;
d) linking the cDNA molecules from step (c) in a second container;
e) combining the linked cDNA molecules from step (d) to produce a library of linked nucleic acid molecules.

36. The method of claim 35, wherein the single cells are B cells, and the percentage of heavy chain variable regions that are correctly paired with the cognate light chain variable regions in the library is increased compared to a method where steps (c) and (d) are performed in the same container.

37. The method of claim 35, wherein the single cells are T cells, and the percentage of T cell receptor alpha chains that are correctly paired with the cognate T cell receptor beta chains in the library is increased compared to a method where steps (c) and (d) are performed in the same container.

38. The method of claim 35, wherein the single cells are NKT cells, and the percentage of T cell receptor alpha chains that are correctly paired with the cognate T cell receptor beta chains in the library is increased compared to a method where steps (c) and (d) are performed in the same container.

39. The method of claim 35, wherein step (d) comprises amplifying and linking the cDNA molecules by overlap extension PCR.

40. The method of claim 39, wherein the overlap extension PCR comprises amplifying the cDNA molecules using one or more internal primers comprising a biotin tag.

41. The method of claim 40, wherein cDNA molecules comprising the biotin tag are removed after step (d).

42. The method of claim 39, wherein the overlap extension PCR comprises amplifying the cDNA molecules using one or more external primers chemically modified to resist nuclease degradation.

43. The method of claim 42, wherein the one or more external primers are chemically modified to include phosphorothioate bonds.

44. The method of claim 43, wherein the cDNA molecules are contacted with a 5′-exonuclease after step (d).

45. A method for producing two or more linked nucleic acid molecules from a single cell, comprising:
(i) isolating a single cell in a first container, and lysing the single cell to release mRNA molecules;
(ii) hybridizing the mRNA molecules to a capture oligonucleotide attached to a solid support, wherein the capture oligonucleotide comprises a sequence complementary to a portion of the mRNA sequence;
(iii) reverse transcribing the mRNA molecules to produce cDNA molecules attached to the solid support in the first container;
(iv) linking the cDNA molecules derived from step (iii) in a second container,
thereby producing linked nucleic acid molecules.

46. The method of claim 45, wherein the capture oligonucleotide further comprises a linker positioned between the solid support and the sequence complementary to a portion of the mRNA sequence.

47. The method of claim 46, wherein the linker is cleaved, releasing the cDNA molecules from the solid support prior to step (iv).

48. The method of claim 45, wherein step (iv) comprises amplifying and linking the cDNA molecules by overlap extension PCR.

49. The method of claim 48, wherein the overlap extension PCR comprises amplifying the cDNA molecules using one or more internal primers comprising a biotin tag.

50. The method of claim 48, wherein cDNA molecules comprising the biotin tag are removed after step (iv).

51. The method of claim 48, wherein the overlap extension PCR comprises amplifying the cDNA molecules using one or more external primers chemically modified to resist nuclease degradation.

52. The method of claim 51, wherein the one or more external primers are chemically modified to include phosphorothioate bonds.

53. The method of claim 52, wherein the cDNA molecules are contacted with a 5′-exonuclease after step (iv).
